(19) United States
(12) Patent Application Publication
(10) Pub. No.: US 2025/0188246 A1
(43) Pub. Date: Jun. 12, 2025

(54) PET DEPOLYMERIZATION PROCESS

(71) Applicant: REPET S.R.L., Roma (IT)

(72) Inventors: Davide Conte, Roma (IT); Simone Di Trapani, Roma (IT)

(21) Appl. No.: 18/846,422

(22) PCT Filed: Mar. 15, 2023
(86) PCT No.: PCT/IB2023/052504

§ 371 Date: Sep. 12, 2024

(30) Foreign Application Priority Data
Mar. 16, 2022 (IT) 102022000005180

(51) Int. Cl. C08J 11/28; B01J 31/02; B01J 35/27; B01J 37/04; B01J 37/08

(52) U.S. Cl. CPC C08J 11/28; B01J 31/0285; B01J 31/0298; B01J 35/27; B01J 37/04; B01J 37/08; B01J 2231/005; C08J 2367/02

(57) ABSTRACT

PET depolymerization process wherein PET, a ionic liquid consisting of (2-hydroxyethyl)trimethylammonium argininate [Cho][Arg] and methanol are mixed together at a temperature ranging from −50 to 70° C. The weight ratio of PET to the ionic liquid is between 10 and 0.05.

### CROSS-REFERENCE TO RELATED APPLICATIONS

This Patent Application claims priority from Italian Patent Application No. 102022000005180 filed on Mar. 16, 2022, the entire disclosure of which is incorporated herein by reference.

### FIELD OF THE ART

The present invention relates to a PET depolymerization process and the preparation of a specific ionic liquid necessary for the depolymerization process.

### BACKGROUND

Polyethylene terephthalate (PET) is one of the most widely used polymeric materials due to its characteristics in terms of electrical properties, chemical resistance, performance at high temperatures, and moulding speed. In this respect, PET has long been the main material used in the food packaging industry. In fact, PET packaging is now also commonly used, in addition to bottles, for producing trays. In particular, features that contribute to making PET an extremely suitable material for food packaging include an efficient recycling loop in almost all continents and high material availability from the recycling loop.

In this respect, polyethylene terephthalate may be disposed of either by chemical recycling or by mechanical recycling. Chemical recycling consists in the depolymerization of the product powder by converting polyethylene terephthalate into the initial raw material.

While chemical recycling allows PET powder to be returned to its monomer state to be subsequently reused for producing PET also for food use, mechanical recycling produces lower quality products of but at the same time is far cheaper. For example, products resulting from mechanical recycling may not be used for the food industry, except for mineral water and soft drinks containers according to special modes.

One way of carrying out chemical recycling relates to depolymerization by glycolysis. Glycolysis is a type of chemical recycling method which converts waste PET into monomer through various chemical reactions. Compared to other recycling methods, the most obvious advantage of glycolysis is that the monomer may be re-polymerized into new PET materials under relatively mild reaction and low-volatile solvent conditions.

In recent years, PET depolymerization processes by glycolysis have been implemented using ionic liquids (ILS) as catalysts. Ionic liquids are organic salts with a low melting point and are considered a safer alternative than traditional organic solvents due to their low vapour pressure and non-flammability. Naturally occurring aliphatic cation ionic liquids, such as (2-hydroxyethyl)trimethylammonium, also known as cholinium, have drawn great interest.

To date, the processes developed for depolymerizing PET using ionic choline liquids provide pressure and temperature conditions such as to make them unattractive when applied on an industrial scale.

The need was therefore felt to make available a process for depolymerizing PET with choline ionic liquid, the technical characteristics of which were such as to make it more industrially attractive than the processes of the prior art.

### OBJECT AND SUMMARY OF THE INVENTION

The inventors of the present invention have implemented a process capable of satisfying the above need.

Two and a half years ago, the inventors devised the PET depolymerization process according to the present invention. At that time, choline-based ionic liquids were beginning to take their first steps by means of publications that focused mainly on their chemical-physical characterisation, which was still lacking in possible applications.

It was only in the spring of 2020 that the first paper was published suggesting the possibility of applying choline-based ionic liquids as catalysts in PET depolymerization processes by means of glycolysis (in which, furthermore, the catalytic function has not been clearly demonstrated). To date, all the publications related to the use of choline-based ionic liquids as catalysts in PET glycolytic depolymerization show no particular developments.

Conversely, during the period from August 2019 to March 2021, the inventors tested their technology firstly with multiple computational simulations (until December 2020) and then through laboratory experiments (until August 2021).

The object of the present invention is a PET depolymerization process, the essential features of which are reported in the independent claim 1, and the secondary and auxiliary features of which are reported in the dependent claims 2-6.

A further object of the present invention is a process for preparing the ionic liquid useful for the depolymerization process according to the present invention, the essential features of which are reported in the independent claim 7, and the secondary and auxiliary features of which are reported in the dependent claims 8 and 9.

A further object of the present invention is a ionic liquid consisting of (2-hydroxyethyl)trimethylammonium argininate [Cho][Arg] made according to the present invention.

### DETAILED DESCRIPTION OF PREFERRED EMBODIMENTS OF THE INVENTION

Embodiments are hereinafter shown for merely illustrative and non-limiting purposes.

### Choline Argininate (Cho-l-Arg) Ionic Liquid Synthesis

0.70 grams of l-arginine powder at the highest available purity were introduced into a 20 ml vial and a suitably sized magnetic stirrer.

1 ml of an aqueous solution of 2-hydroxy-N,N-trimethylethanammonium (ChoOH) was added to the vial drop by drop. The amount of added ChoOH represents a number of moles equal to that of l-arginine. In addition, 7 ml of MilliQ water were also added to the vial. At this point, a constant 350 rpm rotation was set on the magnetic stirrer, while the vial was maintained at a temperature of 30° C. by means of a thermostat bath. These conditions were maintained for a time interval of 48 h.

Adding the ChoOH aqueous solution directly to the l-Arginine powder is necessary to prevent the acid-base equilibrium of the guanidine group of the amino acid, which would make the metathesis reaction between the acid proton of the carboxylic group of the amino acid and the hydroxyl group of the choline hydroxide more difficult.

It was verified that if this addition is carried out drop by drop or in a single solution, on an aqueous solution of l-arginine, maintaining an equal number of moles between l-arginine and choline hydroxide in both solutions, the Cho-l-Arg yield decreased significantly from 100% to about 30%. Subsequently, the contents of the vial were transferred to a 50 mL volume flask.

At this point, the water was removed by means of a rotavapor (pressure values of approx. 35-30 mbars), keeping the water bath first at 30° C. and then, after approx. 40% water removal, at 40° C. The inventors believe that the step of removing water from the reaction matrix must be a time-consuming process which requires great care. From the above, a compound, exceeding from zero to 3.5% by mass, was obtained.

### PET Depolymerization

An example of depolymerization carried out in the laboratory is hereinafter reported.

As may be immediately apparent to a person skilled in the art, the example reported below aims to demonstrate the depolymerization effectiveness of (2-hydroxyethyl)trimethylammonium argininate according to the process of the invention. In this respect, the reported yields are not to be regarded as particularly significant as they may be improved by a cyclic repetition of the process or by industrial adjustments falling the outside characteristics defined in the claims.

9.9936 g of PET from a transparent bottle and cut into pieces of approximately 1×0.5 cm in size and 1.4108 g of ionic liquid consisting of (2-hydroxyethyl)trimethylammonium argininate ([Cho][Arg]) prepared as described above were introduced into a 50 ml reaction flask.

The reaction flask was fixed to a rotavapor with a thermostat bath at 60° C. to allow the viscosity of the ionic liquid to be reduced and the contact with the PET to be increased accordingly. Under these conditions, the rotavapor was activated at a speed of 90 rpm and kept running for 90 minutes.

After 90 minutes, the temperature of the thermostat bath was lowered to 30° C. and the rotavapor was kept in operation for a further 120 minutes.

After the 120 minutes had elapsed, the rotation was stopped and approximately 2 ml of methanol were added drop by drop. Once the addition of methanol was completed, the rotation was reactivated under the same conditions as described above (90 rpm and 30° C.). After 30 minutes of rotation only (without adding methanol) the rotation was stopped and another 2 ml of methanol were added, drop by drop. Once the addition of methanol was completed, the rotation under the above conditions was reactivated for a further 30 minutes.

This operation was repeated 10 times.

According to a further embodiment, an alkali metal methoxide may also be added together with methanol.

Once the methanol additions were completed, the rotation itself was stopped and the non-depolymerized PET was recovered via a buchner funnel and washed with methanol under moderate stirring for 10 minutes. The washing methanol was then recovered and added to the depolymerization matrix consisting of a white precipitate, [Cho][Arg] and methanol.

The non-depolymerized PET was subsequently washed, to be suitably dried and weighed. It was thereby proved that non-depolymerized PET weighed 9.0105 g. A depolymerization yield of about 10% may be inferred from the above.

20 ml of MilliQ water were added to the depolymerization matrix and rotation at 90 rpm was reactivated until the white precipitate was completely dissolved. At this point, the contents of the reaction flask were poured into a beaker together with an additional 150 ml of MilliQ water and a stir bar. The beaker was housed in a crystalliser equipped with an ice bath and the beaker was rotated at a speed of 500 rpm for 15 minutes.

Subsequently, a 1M of HCl solution was added drop by drop to the beaker until the solution in the beaker reached pH 1. Under these conditions, the formation of a white precipitate was noted.

At this point, filtration was performed with a funnel and blue filter paper and the white precipitate was washed thoroughly with more MilliQ water. Once recovered, the white precipitate was dried by placing it in a crystalliser in an oven at a temperature of 94° C. for 1 h.

Once dried, the white compound was submitted to 1 h-nmr, ATR, uv-vis spectrophotometric analysis. As shown by the enclosed spectra, spectrophotometric analysis confirmed that the white compound is terephthalic acid.

The inventors considered that the above reported procedure may be effective at any temperature that keeps the reagents in their liquid state. For this reason, it was considered that in the temperature range from −50 to 70° C., the above reported depolymerization procedure can take place.

In case depolymerization is carried out at a higher temperature than indicated, a number of problems may be involved such as degradation of the (2-hydroxyethyl)trimethylammonium argininate, solubilisation of the monomer reaction product (DMT) within the reaction matrix and subsequent slowing down of the depolymerization process and evaporation of the solvent (CH3OH).

Furthermore, as may be obvious to a person skilled in the art, the use of a higher temperature than indicated necessarily leads to productivity problems if the process of the invention is applied on an industrial scale.

The inventors of the present invention considered the optimal temperature range to be between 20 and 40° C.

Furthermore, the inventors of the present invention believe that the ratio of PET to ionic liquid may be unbalanced with respect to both PET and ionic liquid without affecting the effectiveness of depolymerisation.

### COMPARATIVE EXAMPLES

For comparative purposes, three comparative examples were made in which [Cho][Arg] was replaced with [Cho][Gly], [Cho][Pro] and L-Arginine, respectively.

### Example With [Cho][Gly]

In this example, 1.0830 g of [Cho][Gly] and 1.0980 g of PET were used

The time and temperature conditions of the depolymerization example according to the above invention were repeated, while the amount of methanol added, drop by drop, each time is of 0.1 ml.

[Cho][Gly] was synthesised in the laboratory using the same synthesis procedure as in the case of choline argininate and described above. The reagents used are all Merck-certified.

At the end of the depolymerisation procedure, the recovered non-depolymerized PET was found to weigh 1.0989 g and thus 0.082% more (within the weighing error of the balance) than the PET initially used.

It is clear from the above that the use of the ionic liquid consisting of [Cho][Gly] does not produce any depolymerization.

### Example With [Cho][Pro]

In this example, 1.1620 g of [Cho][Pro] and 1.0452 g of PET were used

The time and temperature conditions of the depolymerization example according to the above invention were repeated, while the amount of methanol added, drop by drop, each time is of 0.1 ml.

[Cho][Pro] was synthesised in laboratory using the same synthesis procedure as in the case of choline argininate and described above. The reagents used are all Merck-certified.

At the end of the depolymerisation procedure, the recovered non-depolymerized PET was found to weigh 1.0511 g and thus 0.0560% more (within the weighing error of the balance) than the PET initially used.

It is clear from the above that the use of the ionic liquid consisting of [Cho][Pro] does not produce any depolymerization.

### Example With L-Arginine

In this example, 0.7084 g L-Arginine and 1.1021 g PET were used.

The time and temperature conditions of the depolymerization example according to the above invention were repeated, while the amount of methanol added, drop by drop, each time is of 0.1 ml.

At the end of the depolymerization procedure, the recovered non-depolymerized PET weighed 1.1022 g.

It is clear from the above that the use of L-Arginine (Merck-certified) does not produce any depolymerization.

From the above description, it appears that, surprisingly, only the use of ionic liquid [Cho][Arg] is able to obtain PET depolymerization under ambient temperature and pressure conditions. Such a result offers important production and environmental advantages. In fact, the process of the present invention allows PET to be depolymerized to obtain the starting monomer, which can, in turn, be polymerized again.

It is important to note that the process of the present invention, when applied to materials comprising PET, succeeds in extracting PET by depolymerization, leaving the other constituent components of the material available.

In this regard, an example of depolymerization of textile fibre carried out in the laboratory is hereinafter reported.

### Example of Textile Fibre Depolymerization

A 50 ml volume flask was loaded with a textile fibre sample consisting of a flap of fabric weighing 0.7158 g (composed of 65% polyester and 35% cotton) and 0.6260 g of ionic liquid consisting of (2-hydroxyethyl)trimethylammonium argininate ([Cho][Arg]). After a blending step carried out at 30° C. between the ionic liquid and the textile fibre using the rotor of a rotavapor, 1 ml of methanol was added over the following nine hours (0.1 ml every hour). Rotation was stopped after each addition to allow methanol to be introduced into the reaction matrix. Subsequently, a further 2.4 ml of methanol was introduced by directly loading 0.4 ml times. The rotor was temporary interrupted at each six addition.

The depolymerization reaction was then interrupted by adding MilliQ water (approximately 50 ml) to the reaction matrix. The residual sample was washed in a beaker with double-distilled water and dried, yielding a weight of 0.5331 g.

The reaction matrix was then filtered by removing cotton polymer residues. The PET monomer was then extracted by additions of 1 M of HCl until pH 1 was reached. The terephthalic acid was collected by filtration, dried in an electric oven and weighed, yielding a mass of 0.14766 g compared to an initial polyester mass of 0.46527 g.

It may therefore be estimated a depolymerization on the PET textile fibre of about 33%.

1. A polyethylene terephthalate (PET) depolymerization process obtained by using a ionic liquid comprising choline; said PET depolymerisation process comprising:
mixing PET, an ionic liquid consisting of (2-hydroxyethyl)trimethylammonium argininate [Cho][Arg], and methanol together at a temperature ranging from 50° C. to 70° C.

2. The PET process according claim 1, wherein a weight ratio between PET and the ionic liquid ranges from 10 to 0.05.

3. The PET process according to claim 1, wherein the mixing includes (a) a first mixing step, during which PET is mixed with the ionic liquid consisting of (2-hydroxyethyl)trimethylammonium argininate [Cho][Arg] at a temperature ranging from 20 to 70° C., and further comprising (b) a depolymerization step, during which the mixture obtained from said mixing step is added to ROH alcohol and stirred at a temperature ranging from −50 to 70° C.

4. The PET process according to claim 1, wherein the methoxide of an alkali metal is added together with the methanol.

5. The PET process according to claim 1, wherein said methanol is added to the mixture drop by drop.

6. The PET process according to claim 3, wherein said depolymerization step comprises the repetition of a methanol adding operation and of a mixing operation one after the other.

7. An ionic liquid preparation process useful for the depolymerization process according to claim 1; said ionic liquid preparation process comprising:
an addition step, during which a choline aqueous solution is added to l-arginine powder; and
a subsequent mixing step, during which the mixture made in the addition step is stirred for a time interval ranging from 12 hours to 72 hours;
said addition step and said mixing step being carried out at a temperature ranging from 15 to 45° C.; the mole ratio between said choline and said l-arginine being between 0.5 and 2.1.

8. The ionic liquid preparation process according to claim 7, wherein said choline aqueous solution is added drop by drop to said arginine powder.

9. The ionic liquid preparation process according to claim 7, further comprising a water removal step following said mixing step; said water removal step taking place at a temperature that is equal to or smaller than 40° C.

10. A ionic liquid consisting of (2-hydroxyethyl)trimethylammonium argininate [Cho][Arg] obtained according to claim 7.
